(19) United States
(12) Patent Application Publication
(10) Pub. No.: US 2018/0071132 A1
(43) Pub. Date: Mar. 15, 2018

(54) MAT STRUCTURE

(71) Applicants: Yi-Chen TSENG, Taichung City (TW); HI-MAX INNOVATION CO,.LTD, Taichung City (TW)

(72) Inventor: Yi-Chen Tseng, Taichung City (TW)

(73) Assignees: HI-MAX INNOVATION CO,.LTD; Yi-Chen TSENG, Taichung City (TW)

(21) Appl. No.: 15/266,749

(22) Filed: Sep. 15, 2016

(51) Int. Cl. A61F 5/14; B32B 3/30; B32B 7/02

(52) U.S. Cl. CPC A61F 5/14; B32B 3/30; B32B 2535/00; B32B 2307/536; B32B 7/02

(57) ABSTRACT

A mat structure includes: an anti-skid layer, a rigid layer, and a soft layer which are superimposed upon one another. A plurality of stimulating protrusions is disposed between the rigid layer and the soft layer to stimulate the ligament and muscle of the foot, which can consequently correct the flatfoot.

### BACKGROUND OF THE INVENTION

### Field of the Invention

The present invention relates to a woven structure, and more particularly to a mat structure.

### Related Prior Art

Flatfoot is a condition in which the arch of the foot has flattened out, and generally includes pseudo flatfoot, flexible flatfoot and rigid flatfoot. Among them, the pseudo flatfoot is common in toddlers due to the fatty of the foot hides the arch formation, and the arch of the foot will gradually be visible as the toddlers grow up. Flexible flatfoot is the most common flatfoot and caused by ligament laxity. Therefore, a lot of therapeutic products for the treatment of flatfoot appeared in the market, for instance, corrective shoes.

However, the flatfoot corrective shoes in the market are very expensive, and the user will feel uncomfortable when wearing the corrective shoes for a long time.

The present invention has arisen to mitigate and/or obviate the afore-described disadvantages.

### SUMMARY

One objective of the present invention is to provide a foot stimulating mat structure which is capable of massaging the foot, relieving pressure and correcting the flatfoot.

Another objective of the present invention is to provide a mat structure capable of simulating various virtual surfaces to provide various stimulations to the foot.

To achieve the above objects, a mat structure in accordance with the present invention comprises: an anti-skid layer, a rigid layer, and a soft layer which are superimposed upon one another.

The anti-skid layer is made of anti-skid material.

The rigid layer has one surface attached to the anti-skid layer.

The soft layer has one surface attached to another surface of the rigid layer and is softer that the rigid layer.

At least one stimulating protrusion is disposed between the rigid layer and the soft layer.

The stimulating protrusion disposed between the rigid and soft layers can stimulate the nerve of the foot to exercise the ligament and muscle of the foot, which consequently corrects the flatfoot.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

FIG. 1 is a cross sectional view of the mat structure in accordance with a preferred embodiment of the present invention;

FIG. 2 is a cross sectional view of the mat structure in accordance with another preferred embodiment of the present invention;

FIG. 3 is an illustrative view showing that a user steps on the mat of the present invention;

FIG. 4A shows that the mat structure in accordance with the present invention simulates a grass land; and

FIG. 4B shows that the mat structure in accordance with the present invention simulates a beach.

### DETAILED DESCRIPTION

The present invention will be clearer from the following description when viewed together with the accompanying drawings, which show, for purpose of illustrations only, the preferred embodiment in accordance with the present invention.

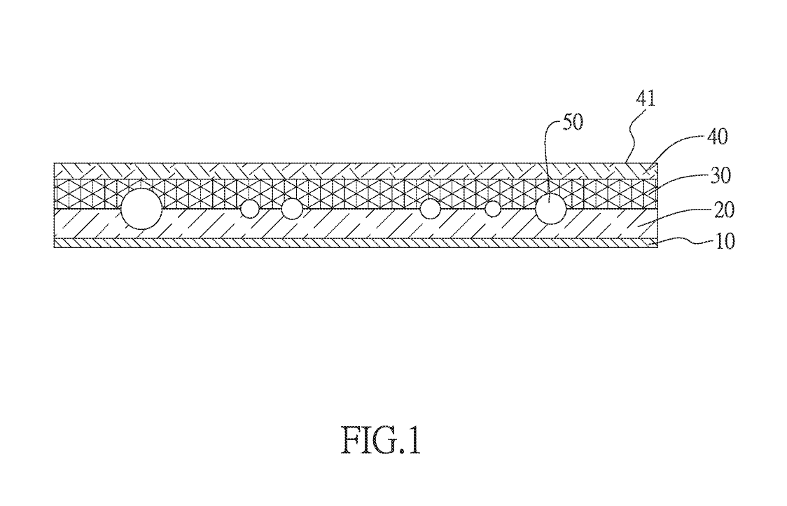

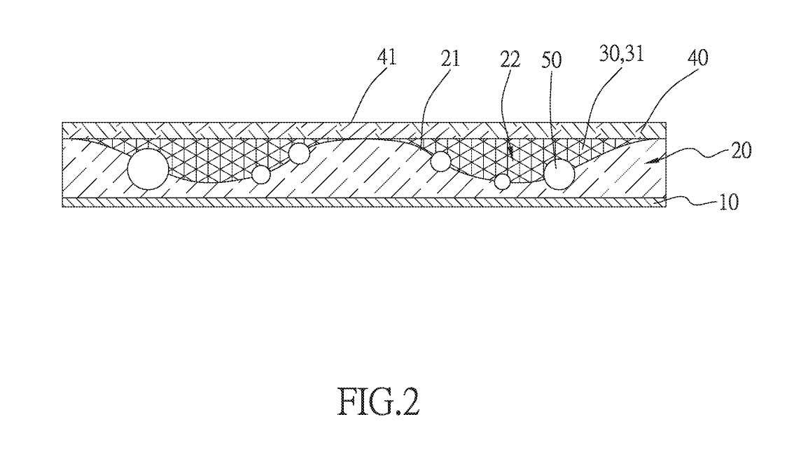

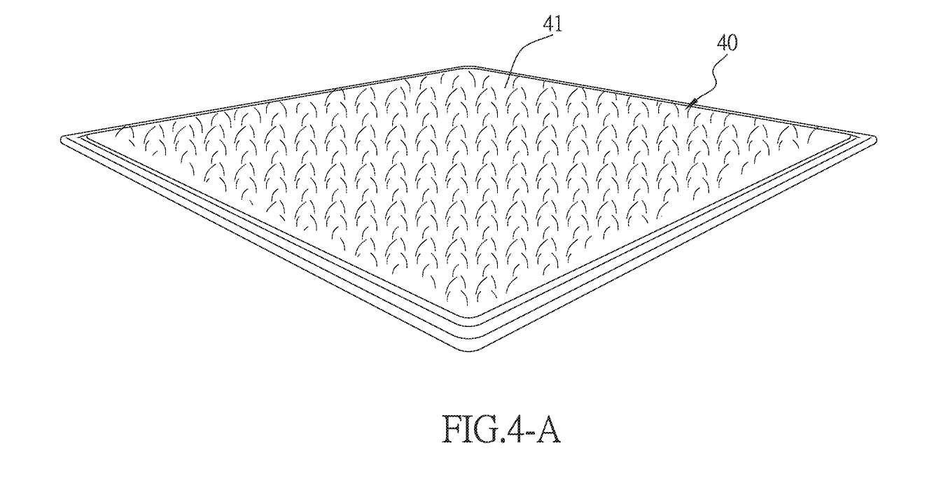

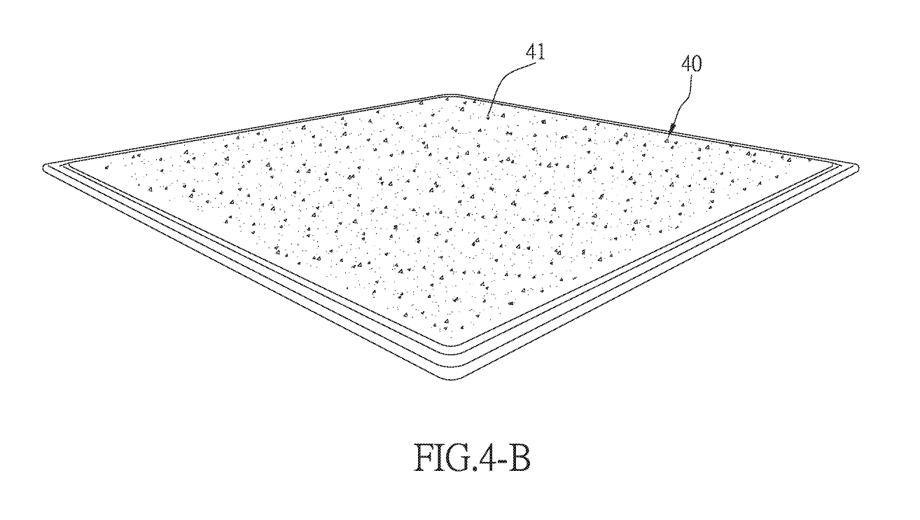

Referring to FIGS. 1-4B, a mat structure in accordance with the present invention comprises: an anti-skid layer 10, a rigid layer 20, a soft layer 30 and a simulation layer 40 which are superimposed upon one another.

The anti-skid layer 10 is made of anti-skid material which can be PVC and rubber.

The rigid layer 20 has one surface attached to the anti-skid layer 10 and has a wavy shape which includes at least one convex portion 21 and at least one concave portion 22.

The soft layer 30 has one surface attached to another surface of the rigid layer 20 and has a wavy shape. The soft layer 30 includes at least one convex portion 31 which is in quantity to, and inserted in, the concave portion 22 of the rigid layer 20. The soft layer 30 is softer that the rigid layer 20.

The simulation layer 40 has one surface attached to another surface of the soft layer 30, and another surface of the simulation layer 40 is a simulation surface 41 which can simulate the touch of beach, grassland and rocky land.

At least one stimulating protrusion 50 is disposed between the rigid layer 20 and the soft layer 30. In this embodiment, the stimulating protrusion 50 has a round shape, and can also be elliptical or polygonal shaped. When there are more than two stimulating protrusions 50, the stimulating protrusions 50 have different sizes.

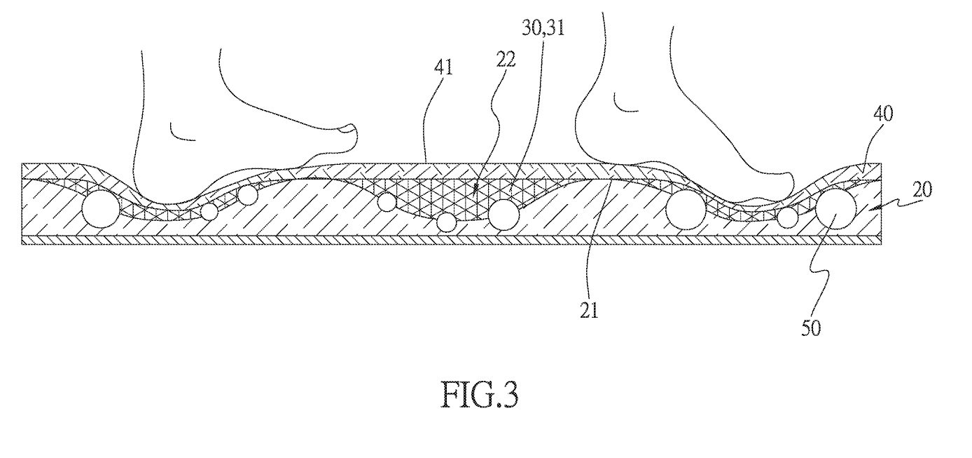

What mentioned above are the structural relations of the main components, and for the operation and function of the embodiment, reference should be made to FIG. 3.

When a user steps on the mat structure of the present invention, the stimulating protrusion 50 can stimulate the ligament and muscle of the foot, so as to correct flatfoot.

It is to be noted that the stimulating protrusion 50 is round shaped to fit the arch of the foot, which is more ergonomic, consequently strengthens the muscle strength of the arch of the foot while reducing the risk of injury.

Preferably, there are plural stimulating protrusions 50 which have different sizes, so as to apply different stimulations to the arch of the foot.

Since the hard rigid layer 20 has a wavy shape and is more rigid than the soft layer 30, so when walking on the mat, the user can stand the stimulating generated by the arch deformation and height change of the convex portion 21 and the concave portion 22, which consequently boosts the strength of the ligament and muscle of the foot. Besides, the rigid layer 20 and the stimulating protrusion 50 are attached to the soft layer 30, which can produce a buffering effect to prevent the occurrence of unnecessary hurt or injury when the user steps directly on the rigid layer 20 or the stimulating protrusions 50.

The mat structure of the present invention is not limited to a tread mat, but can also be used for various applications, such as treadmills or insoles.

While we have shown and described various embodiments in accordance with the present invention, it is clear to those skilled in the art that further embodiments may be made without departing from the scope of the present invention.

What is claimed is:

1. A mat structure, comprising: an anti-skid layer, a rigid layer, and a soft layer which are superimposed upon one another;
the anti-skid layer being made of anti-skid material;
the rigid layer including one surface attached to the anti-skid layer;
the soft layer including one surface attached to another surface of the rigid layer and being softer that the rigid layer; and
at least one stimulating protrusion disposed between the rigid layer and the soft layer.

2. The mat structure as claimed in claim 1 further comprising a simulation layer which has one surface attached to another surface of the soft layer, and another surface of the simulation layer is a simulation surface which can simulate the touch of beach, grassland and rocky land.

3. The mat structure as claimed in claim 1, wherein the stimulating protrusion has a shaped selected from the group consisting of a round shape, an elliptical shape and a polygonal shape, the rigid layer has a wavy shape which includes at least one convex portion and at least one concave portion, and the soft layer has a wavy shape which includes at least one convex portion which is in quantity to, and inserted in, the concave portion of the rigid layer.

4. The mat structure as claimed in claim 1, wherein when there are more than two stimulating protrusions, the stimulating protrusions have different sizes, the rigid layer has a wavy shape which includes at least one convex portion and at least one concave portion, and the soft layer has a wavy shape which includes at least one convex portion which is in quantity to, and inserted in, the concave portion of the rigid layer.

5. The mat structure as claimed in claim 1, wherein the rigid layer has a wavy shape which includes at least one convex portion and at least one concave portion, and the soft layer has a wavy shape which includes at least one convex portion which is in quantity to, and inserted in, the concave portion of the rigid layer.

6. The mat structure as claimed in claim 2, wherein the rigid layer has a wavy shape which includes at least one convex portion and at least one concave portion, and the soft layer has a wavy shape which includes at least one convex portion which is in quantity to, and inserted in, the concave portion of the rigid layer.
